(19) United States
(12) Patent Application Publication
(10) Pub. No.: US 2025/0360102 A1
(43) Pub. Date: Nov. 27, 2025

(54) Compositions and Methods for Radioprotectant/Radiomitigation Hybrid and/or Decorporation

(71) Applicant: Steven BARANOWITZ, Wyncote, PA (US)

(72) Inventor: Steven BARANOWITZ, Wyncote, PA (US)

(21) Appl. No.: 18/874,217

(22) PCT Filed: Jun. 7, 2023
(86) PCT No.: PCT/US2023/024651

§ 371 Date: Dec. 12, 2024

Related U.S. Application Data
(60) Provisional application No. 63/351,837, filed on Jun. 14, 2022.

(51) Int. Cl. A61K 31/404; A61K 33/06; A61K 33/245; A61K 33/26; A61K 33/32; A61K 33/34

(52) U.S. Cl. CPC A61K 31/404; A61K 33/06; A61K 33/245; A61K 33/26; A61K 33/32; A61K 33/34

(57) ABSTRACT

Radioprotectant/radiomitigation hybrid material comprising at least one radiation absorbing component and at least one decorporation component. In particular, the hybrid material is composed of melanin and bismuth. The hybrid material may be formed into a pharmaceutical composition, medical device, protective armour, containers, equipment housing and the like. In another embodiment, a method for determining suitable decorporation materials for radioactive substances is also disclosed.

### CROSS-REFERENCE TO RELATED APPLICATION

This PCT application claims the benefit under 35 U.S.C. § 119(e) of Application Ser. No. 63/351,837 filed on June 14 2022, entitled COMPOSITIONS AND METHODS FOR RADIOPROTECTANT/RADIOMITIGATION HYBRID AND/OR DECORPORATION and whose entire disclosure is incorporated by reference herein.

### SPECIFICATION

### Background

Disclosed herein are methods and compositions comprising radioprotectant/radiomitigation hybrid compositions for simultaneously protecting cells and organisms from radiation and decorporating (removing) radioactive material and metal contamination from the body. The compositions and methods can be formulated, depending on need, as either a device or a drug or a drug-device combination.

There is a great unmet need for ways of preventing and treating radiation from potential dirty radioactive bombs, nuclear blasts, industrial nuclear accidents including meltdowns, etc.

The new modality herein described meets these needs. Fundamentally, it has, for example, two components: (1) radiation absorber and (2) decorporation material.

These two materials tend to be arranged in a variety of geometric ways:

As a spherical, oval, or cylindrical, or rectangular shape, which has a inner (core) component which is the radiation absorber and an outer (shell) component which is the decorporation material.

A bilayer sheet, tile, cup, etc., which has one component side which is the radiation absorber and the other side component which is the decorporation material.

A sandwich, either flat, or curled, which has an inner (core) component which is the radiation absorber and outer (shell) components which are the decorporation material. Other geometric arrangements of the two components are possible, such as:

(a) a chain where the radiation absorbent material alternates with the decorporation material.

(b) a device like an armature, where a central large cylinder or oval is wrapped with coiled strings of the decorporation material.

An example of a radiation absorber component is a biological material such as melanin which has been doped with additives, such as metals (e.g., the metal bismuth) and which on a weight basis absorbs radiation of many types well, and in some cases about as good as lead, or better than lead.

Types of radiation absorbed include all parts of the electromagnetic spectrum such as gamma rays, x-rays, or ultraviolet light, alpha particles, beta particles, electromagnetic pulses, and pressure waves including ultrasound and other vibrations, and radioactive substances such as ²²⁵Actinium, ²²⁷Actinium, ¹³³Barium, ²¹³Bismuth, ¹³⁷Cesium, ⁶⁰Cobalt¹¹¹Indium, ²⁰⁹Lead, ²²⁴Radium, ⁸⁵Strontium, ²²⁸Thorium, Uranium, ⁹⁰Yttrium and metals or other undesirable substances that contaminate organisms.

The decorporation layer may be any material which in the past or future has been demonstrated to bind to those substances which are harmful to the body, and it is wished to remove to preserve health.

One example of a decorporation material is a biological pigment such as melanin which absorbs, adsorbs, or binds in any manner to radioactive substances such as ²²⁵Actinium, ²²⁷Actinium, ¹³³Barium, ²¹³Bismuth, ¹³⁷Cesium, ⁶⁰Cobalt¹¹¹Indium, ²⁰⁹Lead, ²²⁴Radium, ⁸⁵Strontium, ²²⁸Thorium, Uranium, ⁹⁰Yttrium and metals or other undesirable substances that contaminate organisms.

Some of the possible uses of this configuration for a device would be an external shield or armor. For example, in the case of exposure to a dirty bomb the device would have an external decorporation layer to catch radioactive particles, and a internal radiation absorber layer to prevent absorption of the radiation to the body.

Another possible configuration would be as an internal device. For example, a capsule filled with nanoparticles where for each particle the core is the radiation absorber, and the shell is the decorporation material. In certain embodiments, the capsule could be swallowed, and the nanoparticles dispersed throughout the body.

The methods and compositions as disclosed herein could be used either before or after radioactive contamination, e.g., from a dirty radioactive bomb.

If administered before the bomb, the radiation absorber would help protect tissues and the decorporation layer would help quickly get rid of radioactive particles that have come into the body.

If after the bomb, the radiation absorbers would help protect the tissues from radiation coming from radioactive particles already within the body and the decorporation layer would adhere to these particles so they could be excreted.

In the case where a particular organ is affected by contamination, the device or therapeutic could be injected or instilled or implanted into that specific organ.

In certain exemplary embodiments, in the case of inhalation of cobalt-60 particles into the lungs, using bronchoscopy and associated instruments such as catheters, this device or therapeutic could be instilled into the trachea. In this example, cobalt-60 particles are known to localize in the macrophages of the lungs. Melanin particles instilled in the trachea are also known to localize into the macrophages of the lungs. So, these nanoparticles should simultaneously protect tissues against the radiation and also bind to the radioactive particles. This is a form of targeted radiation protection and decorporation at the organ and cellular level.

In certain exemplary embodiments, for example, liver contamination, a catheter could be placed in the portal vein so that the device or therapy would be immediately distributed to the liver.

Being able to shield tissues and organs with a material as protective as lead, on a weight basis, or lighter, and that material being non-toxic, would fill a great unmet need in this area. (Of course, lead, which is an excellent and standard radiation protector, is itself highly toxic.)

Many metal-melanin compounds are thought to be non-toxic, especially bismuth-melanin where both bismuth and melanin are individually non-toxic.

In some formulations the radiation absorber layer and the decorporation layer will remain bound together in the body or on the device. In other applications and formulations, the two layers will dissociate. So, for instance in the case of a central cylinder radiation absorber layer, and an outer coil made of the decorporation material, these two may separate.

The radiation absorber layer may also have decorporation properties. So, for instance, bismuth-melanin may also bind to lithium or strontium.

Historically, research on materials that can perform decorporation has proceeded slowly. Part of the reason is that most of this research has been necessarily conducted in laboratories that have the safety, regulatory, and operational procedures and safeguards which will permit the handling of radioactive isotopes.

It is important to understand that all the isotopes of a single element in the periodic table, have the same chemical structure and behavior in chemical reactions. The different isotopes have different numbers of neutrons, but this does not affect their chemical reactivity.

Therefore, the inventor has discovered that if one wants to determine the ability of a radioactive substance to bind to a candidate decorporation material, one can conduct experiments with the nonradioactive (stable) isotope of that substance and be confident that the radioactive isotope will bind in the same manner and with the same kinetics as the non-radioactive isotope to the candidate decorporation material. While this approach may have been used occasionally in the past, it has never been proposed as a general research method.

Using this method, one can conduct extensive experiments on potential decorporation and shielding materials, in regular laboratories that are not especially outfitted to accommodate all the requirements of handling radioactive materials.

All references cited herein are incorporated herein by reference in their entireties.

### BRIEF SUMMARY

The disclosure provides a radioprotectant/radiomitigation hybrid pharmaceutical composition comprising: at least one therapeutically effective amount of a radiation absorbing component; at least one therapeutically effective amount of a decorporation material component; and at least one pharmaceutically acceptable excipient. The disclosure provides a radioprotectant/radiomitigation hybrid pharmaceutical composition wherein the radiation absorbing component is selected from the group consisting of a biological material, melanin, melanin which has been doped with additives, melanin which has been doped with at least one metal, and combinations thereof. The disclosure provides a radioprotectant/radiomitigation hybrid pharmaceutical composition wherein the radiation absorbing component is melanin which has been doped with at least one metal, wherein the metal is selected from the group consisting of iron, copper, zinc, magnesium, manganese, bismuth, calcium, enamel, cesium, radium, strontium, thorium, uranium, and combinations thereof. The disclosure provides a radioprotectant/radiomitigation hybrid pharmaceutical composition wherein the decorporation material component is selected from the group consisting of a biological pigment, melanin, and combinations thereof. The disclosure provides a radioprotectant/radiomitigation hybrid pharmaceutical composition wherein the decorporation material component absorbs, adsorbs, or binds in any manner to radioactive substances selected from the group consisting of ²²⁵Actinium, ²²⁷Actinium, ¹³³Barium, ²¹³Bismuth, ¹³⁷Cesium, ⁶⁰Cobalt¹¹¹Indium, ²⁰⁹Lead, ²²⁴Radium, ⁸⁵Strontium, ²²⁸Thorium, Uranium, ⁹⁰Yttrium, combinations thereof, beta particles, gamma rays, X-rays, infrared, visible, ultraviolet, the remainder of the electromagnetic spectrum, and metals or other undesirable substances that contaminate organisms, and combinations thereof. The disclosure provides a radioprotectant/radiomitigation hybrid pharmaceutical composition wherein the radioprotectant/radiomitigation hybrid pharmaceutical composition is in the form of a capsule filled with nanoparticles, wherein for each nano particle the core is the radiation absorber, and the shell is the decorporation material. The disclosure provides a radioprotectant/radiomitigation hybrid pharmaceutical composition wherein the radioprotectant/radiomitigation hybrid pharmaceutical composition is in a form that can be injected or instilled or implanted into a specific organ. The disclosure provides a radioprotectant/radiomitigation hybrid pharmaceutical composition wherein the radioprotectant/radiomitigation hybrid pharmaceutical composition is in a form that can be instilled into the lungs, using bronchoscopy. The disclosure provides a radioprotectant/radiomitigation hybrid pharmaceutical composition wherein the radioprotectant/radiomitigation hybrid pharmaceutical composition is in a form that can be delivered into the trachea using catheters. The disclosure provides a radioprotectant/radiomitigation hybrid pharmaceutical composition wherein the radioprotectant/radiomitigation hybrid pharmaceutical composition is in a form that can be injected or instilled or implanted into a catheter could be placed in a portal vein so that the device or therapy would be immediately distributed to the liver. The disclosure provides a radioprotectant/radiomitigation hybrid pharmaceutical composition wherein the radiation absorber layer and the decorporation layer will remain bound together in the body. The disclosure provides a radioprotectant/radiomitigation hybrid pharmaceutical composition wherein the radiation absorber layer and the decorporation layer will dissociate in the body. The disclosure provides a radioprotectant/radiomitigation hybrid pharmaceutical composition wherein the radiation absorber layer and the decorporation layer are in the form of a central cylinder radiation absorber layer, and an outer coil made of the decorporation material, optionally wherein these two layers separate in the body. The disclosure provides a radioprotectant/radiomitigation hybrid pharmaceutical composition wherein the radioprotectant/radiomitigation hybrid pharmaceutical composition is a capsule filled with nanoparticles, that can be swallowed, and the nanoparticles dispersed throughout the body. The disclosure provides a radioprotectant/radiomitigation hybrid pharmaceutical composition wherein the radiation absorber layer also has decorporation properties. The disclosure provides a radioprotectant/radiomitigation hybrid pharmaceutical composition wherein the radioprotectant/radiomitigation hybrid pharmaceutical composition is in a form selected from the group consisting of as a spherical, oval, or cylindrical, or rectangular shape which has a inner (core) component which is the radiation absorber and an outer (shell) component which is the decorporation material.

The disclosure provides a method for the prevention, treatment and/or management of radiation exposure in a subject in need thereof comprising: selecting a subject in need of prevention, treatment and/or management of radiation exposure; administering to the subject the radioprotectant/radiomitigation hybrid pharmaceutical composition as disclosed herein, wherein the administration of the radioprotectant/radiomitigation hybrid pharmaceutical composition prevents, treats, and/or manages radiation exposure in the subject. The disclosure provides a method for the prevention, treatment and/or management of radiation exposure in a subject in need thereof wherein the radiation exposure is a radioactive substances selected from the group consisting of ²²⁵Actinium, ²²⁷Actinium, ¹³³Barium, ²¹³Bismuth, ¹³⁷Cesium, ⁶⁰Cobalt¹¹¹Indium, ²⁰⁹Lead, ²²⁴Radium, ⁸⁵Strontium, ²²⁸Thorium, Uranium, ⁹⁰Yttrium, combinations thereof, beta particles, gamma rays, X-rays, infrared, visible, ultraviolet, the remainder of the electromagnetic spectrum, and metals or other undesirable substances that contaminate organisms, and combinations thereof. The disclosure provides a method for the prevention, treatment and/or management of radiation exposure in a subject in need thereof wherein the radioprotectant/radiomitigation hybrid pharmaceutical composition is in the form of a capsule filled with nanoparticles, wherein for each nano particle the core is the radiation absorber, and the shell is the decorporation material. The disclosure provides a method for the prevention, treatment and/or management of radiation exposure in a subject in need thereof wherein the radioprotectant/radiomitigation hybrid pharmaceutical composition is in a form that could be injected or instilled or implanted into a specific organ. The disclosure provides a method for the prevention, treatment and/or management of radiation exposure in a subject in need thereof wherein the radioprotectant/radiomitigation hybrid pharmaceutical composition is in a form that can be instilled into the lungs, using bronchoscopy. The disclosure provides a method for the prevention, treatment and/or management of radiation exposure in a subject in need thereof wherein the radioprotectant/radiomitigation hybrid pharmaceutical composition is in a form that can be delivered into a trachea using catheters. The disclosure provides a method for the prevention, treatment and/or management of radiation exposure in a subject in need thereof wherein the radioprotectant/radiomitigation hybrid pharmaceutical composition is in a form could be injected or instilled or implanted into a catheter can be placed in the portal vein so that the radioprotectant/radiomitigation hybrid pharmaceutical composition would be immediately distributed to the liver. The disclosure provides a method for the prevention, treatment and/or management of radiation exposure in a subject in need thereof wherein the radiation absorber layer and the decorporation layer will remain bound together in the body. The disclosure provides a method for the prevention, treatment and/or management of radiation exposure in a subject in need thereof wherein the radiation absorber layer and the decorporation layer will dissociate in the body. The disclosure provides a method for the prevention, treatment and/or management of radiation exposure in a subject in need thereof wherein the radiation absorber layer and the decorporation layer are in the form of a central cylinder radiation absorber layer, and an outer coil made of the decorporation material, optionally wherein these two layers separate in the body. The disclosure provides a method for the prevention, treatment and/or management of radiation exposure in a subject in need thereof wherein the radioprotectant/radiomitigation hybrid device is a capsule filled with nanoparticles, that is be swallowed and the nanoparticles dispersed throughout the body. The disclosure provides a method for the prevention, treatment and/or management of radiation exposure in a subject in need thereof wherein the radiation absorber layer also has decorporation properties.

The disclosure provides a kit for the treatment, amelioration, or prevention of radiation exposure, in a patient in need thereof comprising: (a) the radioprotectant/radiomitigation hybrid pharmaceutical composition as disclosed herein; and (b) at least one blister package; a lidded blister; a blister card or packet; a clamshell; an intravenous (IV) package, IV packette or IV container; a bottle; a metal tube; a laminate tube; a plastic tube; a dispenser; a pressurized container; a barrier container; a package; a tray or a shrink wrap, comprising the radioprotectant/radiomitigation hybrid pharmaceutical composition of (a) and instructions for use of the pharmaceutical composition.

The disclosure provides a product of manufacture comprising a blister package; a lidded blister; a blister card or packet; a clamshell; an intravenous (IV) package, IV packette or IV container; a bottle; a metal tube; a laminate tube; a plastic tube; a dispenser; a pressurized container; a barrier container; a package; a tray or a shrink wrap comprising the radioprotectant/radiomitigation hybrid pharmaceutical composition as disclosed herein and instructions for use of the radioprotectant/radiomitigation hybrid pharmaceutical composition.

The disclosure provides a radioprotectant/radiomitigation hybrid device which is implantable into a subject's body, said radioprotectant/radiomitigation hybrid device comprising: at least a radiation absorbing component; at least one decorporation material component. The disclosure provides a radioprotectant/radiomitigation hybrid device which is implantable into a subject's body wherein the radiation absorbing component is selected from the group consisting of a biological material, melanin, melanin which has been doped with additives, melanin which has been doped with at least one metal, and combinations thereof. The disclosure provides a radioprotectant/radiomitigation hybrid device which is implantable into a subject's body wherein the radiation absorbing component is melanin which has been doped with at least one metal, wherein the metal is selected from the group consisting of iron, copper, zinc, magnesium, manganese, bismuth, calcium, enamel, cesium, radium, strontium, thorium, uranium, and combinations thereof. The disclosure provides a radioprotectant/radiomitigation hybrid device which is implantable into a subject's body wherein the decorporation material component is selected from the group consisting of a biological pigment, melanin, and combinations thereof. The disclosure provides a radioprotectant/radiomitigation hybrid device which is implantable into a subject's body wherein the decorporation material component absorbs, adsorbs, or binds in any manner to radioactive substances selected from the group consisting of ²²⁵Actinium, ²²⁷Actinium, ¹³³Barium, ²¹³Bismuth, ¹³⁷Cesium, ⁶⁰Cobalt¹¹¹Indium, ²⁰⁹Lead, ²²⁴Radium, ⁸⁵Strontium, ²²⁸Thorium, Uranium, ⁹⁰Yttrium, combinations thereof, beta particles, gamma rays, X-rays, infrared, visible, ultraviolet, the remainder of the electromagnetic spectrum, and metals or other undesirable substances that contaminate organisms, and combinations thereof. The disclosure provides a radioprotectant/radiomitigation hybrid device which is implantable into a subject's body wherein the radioprotectant/radiomitigation hybrid device is in the form of a capsule filled with nanoparticles, wherein for each nano particle the core is the radiation absorber, and the shell is the decorporation material. The disclosure provides a radioprotectant/radiomitigation hybrid device which is implantable into a subject's body wherein the radioprotectant/radiomitigation hybrid device is in a form that could be injected or instilled or implanted into a specific organ. The disclosure provides a radioprotectant/radiomitigation hybrid device which is implantable into a subject's body wherein the radioprotectant/radiomitigation hybrid device is in a form that can be instilled into the lungs, using bronchoscopy. The disclosure provides a radioprotectant/radiomitigation hybrid device which is implantable into a subject's body wherein the radioprotectant/radiomitigation hybrid device is in a form that can be delivered into the trachea using catheters. The disclosure provides a radioprotectant/radiomitigation hybrid device which is implantable into a subject's body wherein the radioprotectant/radiomitigation hybrid device is in a form could be injected or instilled or implanted into a catheter can be placed in the portal vein so that the device or therapy would be immediately distributed to the liver. The disclosure provides a radioprotectant/radiomitigation hybrid device which is implantable into a subject's body wherein the radiation absorber layer and the decorporation layer will remain bound together in the body or on the device. The disclosure provides a radioprotectant/radiomitigation hybrid device which is implantable into a subject's body wherein the radiation absorber layer and the decorporation layer will dissociate in the body or on the device. The disclosure provides a radioprotectant/radiomitigation hybrid device which is implantable into a subject's body wherein the radiation absorber layer and the decorporation layer are in the form of a central cylinder radiation absorber layer, and an outer coil made of the decorporation material, optionally wherein these two layers separate in the body or on the device. The disclosure provides a radioprotectant/radiomitigation hybrid device which is implantable into a subject's body wherein the radioprotectant/radiomitigation hybrid device is a capsule filled with nanoparticles, that can be swallowed, and the nanoparticles dispersed throughout the body. The disclosure provides a radioprotectant/radiomitigation hybrid device which is implantable into a subject's body wherein the radiation absorber layer also has decorporation properties. The disclosure provides a radioprotectant/radiomitigation hybrid device which is implantable into a subject's body wherein the bismuth-melanin may also bind to lithium or strontium.

The disclosure provides a method for the prevention, treatment, and/or management of radiation exposure in a subject in need thereof comprising selecting a subject in need of prevention, treatment and/or management of radiation exposure; administering to the subject the radioprotectant/radiomitigation hybrid device as disclosed herein, wherein the administration of the radioprotectant/radiomitigation hybrid device prevents, treats and/or manages radiation exposure in the subject. The disclosure provides a method for the prevention, treatment, and/or management of radiation exposure in a subject in need thereof wherein the radiation exposure is a radioactive substances selected from the group consisting of ²²⁵Actinium, ²²⁷Actinium, ¹³³Barium, ²¹³Bismuth, ¹³⁷Cesium, ⁶⁰Cobalt¹¹¹Indium, ²⁰⁹Lead, ²²⁴Radium, ⁸⁵Strontium, ²²⁸Thorium, Uranium, ⁹⁰Yttrium, combinations thereof, beta particles, gamma rays, X-rays, infrared, visible, ultraviolet, the remainder of the electromagnetic spectrum, and metals or other undesirable substances that contaminate organisms, and combinations thereof. The disclosure provides a method for the prevention, treatment, and/or management of radiation exposure in a subject in need thereof wherein the radioprotectant/radiomitigation hybrid device is in the form of a capsule filled with nanoparticles, wherein for each nano particle the core is the radiation absorber, and the shell is the decorporation material. The disclosure provides a method for the prevention, treatment, and/or management of radiation exposure in a subject in need thereof wherein the radioprotectant/radiomitigation hybrid device is in a form that could be injected or instilled or implanted into a specific organ.

The disclosure provides a method for the prevention, treatment, and/or management of radiation exposure in a subject in need thereof wherein the radioprotectant/radiomitigation hybrid device is in a form that can be instilled into the lungs, using bronchoscopy. The disclosure provides a method for the prevention, treatment, and/or management of radiation exposure in a subject in need thereof wherein the radioprotectant/radiomitigation hybrid device is in a form that can be delivered into the trachea using catheters. The disclosure provides a method for the prevention, treatment, and/or management of radiation exposure in a subject in need thereof wherein the radioprotectant/radiomitigation hybrid device is in a form that can be injected or instilled or implanted into a catheter could be placed in the portal vein so that the device or therapy would be immediately distributed to the liver. The disclosure provides a method for the prevention, treatment, and/or management of radiation exposure in a subject in need thereof wherein the radiation absorber layer and the decorporation layer will remain bound together in the body or on the device. The disclosure provides a method for the prevention, treatment, and/or management of radiation exposure in a subject in need thereof wherein the radiation absorber layer and the decorporation layer will dissociate in the body or on the device. The disclosure provides a method for the prevention, treatment, and/or management of radiation exposure in a subject in need thereof wherein the radiation absorber layer and the decorporation layer are in the form of a central cylinder radiation absorber layer, and an outer coil made of the decorporation material, optionally wherein these two layers separate in the body or on the device. The disclosure provides a method for the prevention, treatment, and/or management of radiation exposure in a subject in need thereof wherein the radioprotectant/radiomitigation hybrid device is a capsule filled with nanoparticles, that is be swallowed and the nanoparticles dispersed throughout the body. The disclosure provides a method for the prevention, treatment, and/or management of radiation exposure in a subject in need thereof wherein the radiation absorber layer also has decorporation properties.

The disclosure provides a radioprotectant/radiomitigation hybrid material comprising: at least a radiation absorbing component; at least one decorporation material component. The disclosure provides a radioprotectant/radiomitigation hybrid material wherein the radiation absorbing component is selected from the group consisting of a biological material, melanin, melanin which has been doped with additives, melanin which has been doped with at least one metal, and combinations thereof. The disclosure provides a radioprotectant/radiomitigation hybrid material wherein the radiation absorbing component is melanin which has been doped with at least one metal, wherein the metal is selected from the group consisting of iron, copper, zinc, magnesium, manganese, bismuth, calcium, enamel, cesium, radium, strontium, thorium, uranium, and combinations thereof. The disclosure provides a radioprotectant/radiomitigation hybrid material wherein the decorporation material component is selected from the group consisting of a biological pigment, melanin, and combinations thereof. The disclosure provides a radioprotectant/radiomitigation hybrid material wherein the decorporation material component absorbs, adsorbs, or binds in any manner to radioactive substances selected from the group consisting of ²²⁵Actinium, ²²⁷Actinium, ¹³³Barium, ²¹³Bismuth, ¹³⁷Cesium, ⁶⁰Cobalt¹¹¹Indium, ²⁰⁹Lead, ²²⁴Radium, ⁸⁵Strontium, ²²⁸Thorium, Uranium, ⁹⁰Yttrium, combinations thereof, beta particles, gamma rays, X-rays, infrared, visible, ultraviolet, the remainder of the electromagnetic spectrum, and metals or other undesirable substances that contaminate organisms, and combinations thereof. The disclosure provides a radioprotectant/radiomitigation hybrid material wherein the radioprotectant/radiomitigation hybrid device is in a form selected from the group consisting of a bilayer sheet, tile, cup, etc., which has one component side which is the radiation absorber and the other side component which is the decorporation material. The disclosure provides a radioprotectant/radiomitigation hybrid material the wherein radioprotectant/radiomitigation hybrid device is in a form selected from the group consisting of a sandwich, either flat, or curled, which has an inner (core) component which is the radiation absorber and outer (shell) components which are the decorporation material. The disclosure provides a radioprotectant/radiomitigation hybrid material wherein the radioprotectant/radiomitigation hybrid device is in a form selected from the group consisting of a chain where the radiation absorbent material alternates with the decorporation material; a device which is an armature, where a central large cylinder or oval is wrapped with coiled strings of the decorporation material. The disclosure provides a radioprotectant/radiomitigation hybrid material wherein the radiation absorber layer also has decorporation properties. The disclosure provides a radioprotectant/radiomitigation hybrid material wherein the bismuth-melanin may also bind to lithium or strontium. The disclosure provides a radioprotectant/radiomitigation hybrid material wherein the radiation absorbing material and the decorporation material are present in a ratio selected from the group consisting of about 1 to about 15, about 15 to about 1, about 1 to about 10, about 10 to about 1, about 1 to about 5, about 5 to about 1, about 2 to about 98, about 5 to about 95, about 20 to about 80, about 30 to about 70, about 40 to about 60, about 50 to about 50, about 60 to about 40 about 70 to about 30 about 80 to about 20, about 95 to about 5, and about 98 to about 2 (percent by weight). The disclosure provides a radioprotectant/radiomitigation hybrid material wherein the material is formed into an article which is an item selected from the group consisting of helmets, body armor, vehicle armor, aircraft armor, watercraft armor, structure armor, equipment housing, blast protection panels, ballistic protection panels, and cargo containers. The disclosure provides a radioprotectant/radiomitigation hybrid material, further comprising at least one additive material selected from the group consisting of process aids, modifiers, colorants, fibers, adhesion promoters and fillers. The disclosure provides a radioprotectant/radiomitigation hybrid material wherein the material is formed into an article which is an item selected from the group consisting of helmets, body armor, vehicle armor, aircraft armor, watercraft armor, structure armor, equipment housing, blast protection panels, ballistic protection panels and cargo containers.

The disclosure provides a process for forming a radioprotectant/radiomitigation hybrid material comprising mixing: at least a radiation absorbing component; at least one decorporation material component; and optionally, at least one binder; and shaping the composite material into an article. The disclosure provides a process for forming a radioprotectant/radiomitigation hybrid material wherein the radiation absorbing component is selected from the group consisting of a biological material, melanin, melanin which has been doped with additives, melanin which has been doped with metals, and combinations thereof. The disclosure provides a process for forming a radioprotectant/radiomitigation hybrid material wherein the radiation absorbing component is melanin which has been doped with at least one metal, wherein the metal is selected from the group consisting of iron, copper, zinc, magnesium, manganese, bismuth, calcium, enamel, cesium, radium, strontium, thorium, uranium, and combinations thereof. The disclosure provides a process for forming a radioprotectant/radiomitigation hybrid material wherein the decorporation material component is selected from the group consisting of a biological pigment, melanin, and combinations thereof. The disclosure provides a process for forming a radioprotectant/radiomitigation hybrid material wherein the decorporation material component absorbs, adsorbs, or binds in any manner to radioactive substances selected from the group consisting of ²²⁵Actinium, ²²⁷Actinium, ¹³³Barium, ²¹³Bismuth, ¹³⁷Cesium, ⁶⁰Cobalt¹¹¹Indium, ²⁰⁹Lead, ²²⁴Radium, ⁸⁵Strontium, ²²⁸Thorium, Uranium, ⁹⁰Yttrium, combinations thereof, beta particles, gamma rays, X-rays, infrared, visible, ultraviolet, the remainder of the electromagnetic spectrum, and metals or other undesirable substances that contaminate organisms, and combinations thereof. The disclosure provides a process for forming a radioprotectant/radiomitigation hybrid material wherein the step of shaping comprises using a technique selected from the group consisting of molding, compression molding, stamping, bending, thermoforming, injection molding, additive manufacturing, coining, and extruding. The disclosure provides a process for forming a radioprotectant/radiomitigation hybrid material wherein the radiation absorbing material and the decorporation material are mixed using a machine selected from the group consisting of a single screw extruder, a counter-rotating twin-screw extruder, a co-rotating twin-screw extruder, a Henschel mixer, and a cokneader. The disclosure provides a process for forming a radioprotectant/radiomitigation hybrid material wherein the radiation absorbing material and the decorporation material are present in a ratio selected from the group consisting of about 1 to about 15, about 15 to about 1, about 1 to about 10, about 10 to about 1, about 1 to about 5, about 5 to about 1, about 2 to about 98, about 5 to about 95, about 20 to about 80, about 30 to about 70, about 40 to about 60, about 50 to about 50, about 60 to about 40 about 70 to about 30 about 80 to about 20, about 95 to about 5, and about 98 to about 2 (percent by weight). The disclosure provides a process for forming a radioprotectant/radiomitigation hybrid material wherein the material is formed into an article which is an item selected from the group consisting of helmets, body armor, vehicle armor, aircraft armor, watercraft armor, structure armor, equipment housing, blast protection panels, ballistic protection panels, and cargo containers.

The disclosure provides a method for determine the ability of a radioactive substance to bind to a candidate decorporation material, the method comprising the steps of: providing at least one nonradioactive (stable) isotope of a test material; providing a decorporation material to be tested; mixing the at least one nonradioactive (stable) isotope of a test material and the decorporation material to be tested; measuring the amount of at least one nonradioactive (stable) isotope of a test material absorbed by the decorporation material. The disclosure provides a method for determine the ability of a radioactive substance to bind to a candidate decorporation material wherein the radiation absorbing component is selected from the group consisting of a biological material, melanin, melanin which has been doped with additives, melanin which has been doped with at least one metal, and combinations thereof. The disclosure provides a method for determine the ability of a radioactive substance to bind to a candidate decorporation material wherein the radiation absorbing component is melanin which has been doped with at least one metal, wherein the metal is selected from the group consisting of iron, copper, zinc, magnesium, manganese, bismuth, calcium, enamel, cesium, radium, strontium, thorium, uranium, and combinations thereof. The disclosure provides a method for determine the ability of a radioactive substance to bind to a candidate decorporation material wherein the decorporation material component is selected from the group consisting of a biological pigment, melanin, and combinations thereof. The disclosure provides a method for determine the ability of a radioactive substance to bind to a candidate decorporation material wherein the decorporation material component absorbs, adsorbs, or binds in any manner to at least one nonradioactive (stable) isotope of a test material of a radioactive substances selected from the group consisting of ²²⁵Actinium, ²²⁷Actinium, ¹³³Barium, ²¹³Bismuth, ¹³⁷Cesium, ⁶⁰Cobalt¹¹¹Indium, ²⁰⁹Lead, ²²⁴Radium, ⁸⁵Strontium, ²²⁸Thorium, Uranium, ⁹⁰Yttrium, combinations thereof, beta particles, gamma rays, X-rays, infrared, visible, ultraviolet, the remainder of the electromagnetic spectrum, and metals or other undesirable substances that contaminate organisms, and combinations thereof.

The disclosure provides for the use of the compositions of the disclosure for the production of a medicament for preventing and/or treating the indications as set forth herein.

In accordance with a further embodiment, the present disclosure provides a use of the pharmaceutical compositions described above, in an amount effective for use in a medicament, and most preferably for use as a medicament for treating a disease or disorder, for example, as set forth in herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### DETAILED DESCRIPTION

Radioactivity is a characteristic of a material and radiation is the result of a radioactive material, often a naturally occurring or artificially generated material, or sometimes may be artificially generated radiation such as from an X-ray tube. The result of radioactivity is the generation particle emissions, including neutron, x-ray, alpha, beta or gamma particles. Alpha particles are considered high linear energy transfer (LET) particles and deliver substantive damage to DNA in the form of double stranded DNA breaks, which are very difficult for cells to repair properly. Gamma rays, and x rays, in contrast are low LET particles and operate by the generation of radiolysis of water generating hydroxyl free radicals in the vicinity of DNA causing single strand and double stranded breaks following a linear-quadratic curve of cell survival v. dose, culminating in a loss of reproductive integrity of the cancer cells Likewise beta particles, though low in linear energy transfer can cause double stranded breaks and destroy DNA through clusters of single stranded breaks which can be made permanent by oxygen fixation in non-hypoxic environments.

The radiation produced by radioactive materials provides a low-cost way to disinfect food, sterilize medical equipment, treat certain kinds of cancer, find oil, build sensitive smoke detectors, and provide other critical services in our economy. As a result, significant amounts of radioactive materials are stored in laboratories, food irradiation plants, oil drilling facilities, medical centers, and many other sites. As such, radioactive materials are available for both their beneficial uses, and for non-beneficial uses such as terrorism or nuclear warfare, although the latter may be less dependent on fission or fusion reaction. Delivery of a general radiation releasing event such as an atomic bomb requires a set of complex delivery systems that decrease the probability that this type of device would be delivered by a terrorist. However, concern over the creation of what is termed a “dirty bomb” has received an increasingly high priority for response. As described by the Center for Disease Control website, a dirty bomb is a mix of explosives, such as dynamite, with radioactive powder or pellets. When the dynamite or other explosives are set off, the blast carries radioactive material into the surrounding area. A “dirty bomb” is created by combining radioactive material and a conventional explosive such as dynamite to aerosolize the material in the local environment. This type of device has also been called a radiological dispersal device (RDD). A number of materials are considered as candidates for use in a RDD based on either availability from industrial sources or other characteristics. As an example, the world-wide use of the radiation releasing material Cobalt-60 (Co-60 or 60Co) in conventional and medical applications is often cited as an example of a material that could be used to form a dirty bomb. Strontium (Sr)-90, yttrium (Y)-90, cesium (Cs)-137, iridium (Ir)-192, americium (Am)-241, iodine (I)-125 and 131, uranium (U)-233, 234, 235, and 238, plutonium (Pu)-239, radium (Ra)-226, tritium (hydrogen-3 or H-3), phosphorus (P)-32 and palladium (Pd)-103 would generate the same kind of dirty bomb effect.

Breathing or swallowing the aerosolized dust from an RDD explosion can result in the inhalation and ingestion of radioactive particles. As the amount of material ingested or inhaled is likely to be less than that expected to cause immediate death, the damage from such exposures is likely to be due to the effects of prolonged exposure to the radiation releasing material in close proximity to the cells of the body, while inside the body. In contrast to an external exposure, which is brief, ingestion, inhalation or systemic intake of radioactive material results in a prolonged exposure, increasing the likelihood of damage to the body. The more quickly a radioactive material such as Co-60 or an RDD Radiotoxic Material is removed from the body, the fewer and less serious the health effects will be. A corollary to this observation is the concept that the longer the radioactive material stays in the body, the more difficult it becomes to remove the material. Further, the longer the agent is in the body, the more prolonged its side effects, including side effects related to systemic stress as opposed to the radioactivity per se, e.g., while initially, the damage is likely directly from ionization resulting from radioactive decay, as time passes, relatively more damage will result from combinations of impaired system functions or immune functions which functional impairments were caused by initial ionization damage. Materials and methods that facilitate the removal of radiation producing materials from the body fall in the class of “decorporation.” The broader class of nuclear, biological and chemical weapons are referred to as nuclear, biological and chemical (“NBC”) weapons.

As used herein the term “active pharmaceutical ingredient” (“API”) or “pharmaceutically active agent” is a drug or agent which can be employed as disclosed herein and is intended to be used in the human or animal body in order to heal, to alleviate, to prevent or to diagnose diseases, ailments, physical damage or pathological symptoms; allow the state, the condition or the functions of the body or mental states to be identified; to replace active substances produced by the human or animal body, or body fluids; to defend against, to eliminate or to render innocuous pathogens, parasites or exogenous substances or to influence the state, the condition or the functions of the body or mental states. Drugs in use can be found in reference works such as, for example, the Rote Liste or the Merck Index. Examples which may be mentioned include, for example, tretinoin.

As used herein, “pharmaceutically acceptable salts” refer to derivatives of the disclosed compounds wherein the therapeutic compound is modified by making acid or base salts thereof. Examples of pharmaceutically acceptable salts include, but are not limited to, mineral or organic acid salts of the active agent. The pharmaceutically acceptable salts include the conventional non-toxic salts, for example, from non-toxic inorganic or organic acids. For example, such conventional non-toxic salts include those derived from inorganic acids such as hydrochloric, hydrobromic, sulfuric, sulfonic, sulfamic, phosphoric, nitric and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and other known to those of ordinary skill in the pharmaceutical sciences. Lists of suitable salts are found in texts such as Remington's Pharmaceutical Sciences, 18th Ed. (Alfonso R. Gennaro, ed.; Mack Publishing Company, Easton, Pa., 1990); Remington: the Science and Practice of Pharmacy 19th Ed. (Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are hereby incorporated by reference.

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a chicken, horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic agents).

As used herein, the terms “therapies” and “therapy” can refer to any method(s), composition(s), and/or agent(s) that can be used in the prevention, treatment and/or management of a disease or condition, or one or more symptoms thereof.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, and/or the amelioration of one or more symptoms thereof resulting from the administration of one or more therapies.

As used herein, the term “multi-particulates” refers to one or more unit dosage systems such as, but not limited to, pellets, beads, spheres, mini-tablets, seeds, spheroids or granules with modified drug release profile. The multi-particulates comprise a drug-release controlling and/or drug-protecting film or matrix, such as a polymeric film or matrix, whose intactness or efficiency is susceptible to certain conditions such as heat or mechanical forces that may occur during post-processing. The expression “core material” describes the nature of the interior part of multi-particulates that may also comprise a functional coat. Exemplary “core-materials” may be pellets (spherical matrix systems that contain a drug and further excipients), granules (less spherical particles that are almost entirely composed of drug) or nonpareils (spherical particles without drug).

As used herein, the term “about” when used in conjunction with a stated numerical value or range has the meaning reasonably ascribed to it by a person skilled in the art, i.e., denoting somewhat more or somewhat less than the stated value or range.

### Radioprotection

Radioprotection measures, those timed before radiation exposure, are in all cases the most direct and effective approaches. Radioprotection entails shielding, shelter, avoidance, protection and economy. Radioprotection involves reduction of free radical damages through cooling, hypoxia, increased endogenous antioxidant levels, enhanced exogenous free radical scavengers and antioxidants. Radioprotection anti-free radical agents reduce the damages from the mixture of free radicals generated intracellularly by ionizing radiation--such as peroxyl free radicals and hydroxyl free radicals. Sequestration of free radicals prevents multitudes of secondary damages to DNA nucleic acids, proteins and lipids, such as hematopoietic devastation as evidenced by leucopenia, lymphocytopenia, altered immune cell profiles and abnormal cellularity of the thymus and spleen as well as skin and mucous membrane damage and RIBEs, and later chronic inflammation and fibrotic sequelae and cancer. Radiomitigation treatments, in certain embodiments, are measures taken before, during and/or after exposure to radiation, and may be administered before, during, and/or after symptoms of radiation exposure. Radiorecovery refers to, in certain embodiments, the employment of typical medical interventions for radiation damage symptoms, including inflammatory and fibrotic sequelae and cancer. DNA protection treatments lessen the severity of DNA damage through various mechanisms such as DNA compaction. DNA protectors prevent DNA damages by alterations of histones, topoisomerase, polyamines, cell cycling and mitosis. For example, DNA compaction such as is achieved by Sea Buckthorn radioprotects against gamma radiation and X-rays DNA single and double strand breaks; after the danger has passed the compaction subsides for necessary DNA repair mechanisms to resume. Thus, timing of such agent use is critical. General radioprotectant soften benefit against radiation damage via many mechanisms Timing of treatments is critical because the impact of preventative measures far outweighs symptomatological treatments.

### Radioprotectant/Radiomitigation Hybrid

The Radioprotectant/Radiomitigation Hybrid as disclosed herein comprises at least one component which is a radioprotection component which entails shielding, shelter, avoidance, protection and economy. Radioprotection involves reduction of free radical damages through cooling, hypoxia, increased endogenous antioxidant levels, enhanced exogenous free radical scavengers and antioxidants. Radioprotection anti-free radical agents reduce the damages from the mixture of free radicals generated intracellularly by ionizing radiation--such as peroxyl free radicals and hydroxyl free radicals. Sequestration of free radicals prevents multitudes of secondary damages to DNA nucleic acids, proteins and lipids, such as hematopoietic devastation as evidenced by leucopenia, lymphocytopenia, altered immune cell profiles and abnormal cellularity of the thymus and spleen as well as skin and mucous membrane damage and RIBEs, and later chronic inflammation and fibrotic sequelae and cancer. The Radioprotectant/Radiomitigation Hybrid as disclosed herein comprises at least one component which is a radiomitigation component, which in certain embodiments, are measures taken before, during, and/or after exposure to radiation, and may be administered before, during, and/or after symptoms of radiation exposure. Radiorecovery refers to, in certain embodiments, the employment of typical medical interventions for radiation damage symptoms, including inflammatory and fibrotic sequelae and cancer

### Decorporation

Materials and methods that facilitate the removal of radiation producing materials from the body fall in the class of “decorporation.” Preemptive anticorporation radioprotection, pre-arms against uptake of exposures to radioisotopes. Anticorporation means are pharmacologic ways to competitively inhibit the uptake of certain radioisotopes into body depots using the same or similar non-radioactive elements or drugs which prevent uptake in other ways. For example, I-127 is a stable similar non-radioactive element for unstable highly radioactive I-131; stable calcium is a similar non-radioactive element for Sr-90.

### Radiomimetic Protectants

Radiomimetics are compounds which generate damages similar to ionizing radiation. Chemicals known as peroxides act as radiomimetics by causing free radical damages. During tooth whitening procedures employing peroxides, hydroxyl and other free radicals are generated in gingival tissues. These free radicals are capable of causing mucosal damage as ionizing radiation-caused free radicals, including DNA mutations which pose a theoretical concern of carcinogenicity and oral cancer. Peroxides are recognized as tumor promoters, irritants and cytotoxins. Hydrogen peroxide at concentrations over 10% is a corrosive. The US FDA approves the sale of dental gels that are under 6% hydrogen peroxide or under 16% carbamide peroxide, however some dental practices employ up to 25% hydrogen peroxide. The anti-free-radical mixture of the invention is of use as a preemptive therapeutic radioprotective.

### Radiomitigation

Radiomitigation, in certain embodiments herein, entails treatments taken before, during and/or after exposure to radiation, and may be administered before, during, and/or after symptoms of radiation exposure. The radiomitigation measures may include decorporation of radioisotopes of the invention Once radiocontaminants embed in various body organs and structures they emit ionizing radiation without benefit of distance, constantly, typically with long half-lives, and without easy means of removal. Decorporation measures geared to clearing radiocontamination can improve health; this was seen with children in Chernobyl fed apple pectin showed greatly reduced cesium-137, which was unavoidable in local produce. Decorporation effects enhanced excretion and removal of a radiocontaminant whether internal or external, from the body primarily via sweat, feces, and urine, and combinations achieve synergistic increases in clearance. Therefore, decorporation agents may include substances and methods for enhanced removal via bathing, rinsing, sweating, wiping, diuretics and purgatives which use a variety of mechanisms such as pH alterations, adsorption, binding, chelation, precipitation, enhanced GFR and catharsis of the invention. For example, cobalt-60 and other radioisotopes are absorbed through the GI, thus prompt removal from the tract can prevent a large degree of disastrous whole body irradiation symptoms. Recent ingestion of radioisotopes can be medically treated with substances which prevent systemic uptake such as purgatives, anti-absorptives, binders, adsorbents, chelators and acids/bases. Treatments may be needed for long term Delayed radiomitigation agents such as in the present invention. These include some antioxidant measures which have been shown to work after radiation exposure. Later, delayed use of specific agents are merited, such as enhanced DNA repair and anti-RIBEs (radiation-induced bystander effect) agents.

### Radiorecovery

Radiorecovery treatments include medical treatments which work towards anti-infection, bone-marrow and spleen recovery, burn therapies and survival as in the present invention. Radiorecovery medical interventions are for acute radiation symptoms, addressing such needs as hematopoietic damage, immune devastation, sepsis, nausea, vomiting, diarrhea, pain, contamination and death. There are no real prescribed medical-algorithms for low-dose radiation exposures and few for high dose exposures. In fact aside from amifostine and the soon-to-be-approved Ex-Rad, drugs utilized in any setting for radioprotection and radiomitigation will mostly be “off label” applications; the same situation holds true for herbs, minerals, and vitamins and even in emergency settings.

### Melanin

As used here, the term “melanin” refers to melanins, melanin precursors, melanin analogs, melanin variants, melanin derivatives, and melanin-like pigments, unless the context dictates otherwise. The term “melanin-like” also refers to hydrogels with melanin-like pigmentation and quinoid electrophilicity. This electrophilicity can be exploited for facile coupling with biomolecules.

As used herein, the term “melanin analog” refers to a melanin in which a structural feature that occurs in naturally-occurring or enzymatically-produced melanins is replaced by a substituent divergent from substituents traditionally present in melanin. An example of such a substituent is a selenium, such as selenocysteine, in place of sulfur.

As used herein, the term “melanin derivative” refers to any derivative of melanin which is capable of being converted to either melanin or a substance having melanin activity. An example of a melanin derivative is melanin attached to a dihydrotrigonelline carrier such as described in Bodor, N., Ann. N. Y. Acad. Sci. 507, 289 (1987), which enables the melanin to cross the blood-brain barrier. The term melanin derivatives is also intended to include chemical derivatives of melanin, such as an esterified melanin.

As used herein, the term “melanin variant” refers to various subsets of melanin substances that occur as families of related materials. Included in these subsets, but not limited thereto, are:

(1) Naturally occurring melanins produced by whole cells that vary in their chemical and physical characteristics; (2) Enzymatically produced melanins prepared from a variety of precursor substrates under diverse reaction conditions; (3) Melanin analogs in which a structural feature that occurs in (1) or (2) above is replaced by an unusual substituent divergent from the traditional; and (4) Melanin derivatives in which a substituent in a melanin produced in (1), (2) or (3) above is further altered by chemical or enzymatic means.

As used herein, the term “Melanin-like substances” refers to heteropolymers of 5-6-dihydroxyindole and 5-6-dihydroxyindole-2-carboxylic acid which have one or more properties usually associated with natural melanins, such as UV absorption or semiconductor behavior.

The melanins comprise a family of biopolymer pigments. A frequently used chemical description of melanin is that it is comprised of “heteropolymers of 5-6-dihydroxyindole and 5-6-dihydroxyindole-2-carboxylic acid” (Bettinger et al., 2009). Melanins are polymers produced by polymerization of reactive intermediates. The polymerization mechanisms include, but are not limited to, autoxidation, enzyme-catalyzed polymerization and free radical initiated polymerization. The reactive intermediates are produced chemically, electrochemically, or enzymatically from precursors. Suitable enzymes include, but are not limited to, peroxidases, catalases, polyphenol oxidases, tyrosinase, tyrosine hydroxylases, and laccases. The precursors that are connected to the reactive intermediates are hydroxylated aromatic compounds. Suitable hydroxylated aromatic compounds include, but are not limited to 1) phenols, polyphenols, aminophenols and thiophenols of aromatic or polycyclicaromatic hydrocarbons, including, but not limited to, phenol, tyrosine, pyrogallol, 3-aminotyrosine, thiophenol and a-naphthol; 2) phenols, polyphenols, aminophenols, and thiophenols of aromatic heterocyclic or heteropoly cyclic hydrocarbons such as, but not limited to, 2-hydroxypyrrole,4-hydroxy-1,2-pyrazole, 4-hydroxypyridine, 8-hydroxyquinoline, and 4,5-dihydroxybenzothiazole.

The term melanin includes naturally occurring melanin polymers as well as melanin analogs as defined below. Naturally occurring melanins include eumelanins, phaeomelanins, neuromelanins and allomelanins.

As used here, the term “melanin” refers to melanins, melanin precursors, melanin analogs, melanin variants, melanin derivatives, melanin-like pigments, and/or melanosomes, unless the context dictates otherwise. The term “melanin-like” also refers to hydrogels with melanin-like pigmentation and quinoid electrophilicity. This electrophilicity can be exploited for facile coupling with biomolecules.

As used herein, the term “melanin analog” refers to a melanin in which a structural feature that occurs in naturally-occurring or enzymatically-produced melanins is replaced by a substituent divergent from substituents traditionally present in melanin. An example of such a substituent is a selenium, such as selenocysteine, in place of sulfur.

As used herein, the term “melanin derivative” refers to any derivative of melanin which is capable of being converted to either melanin or a substance having melanin activity. An example of a melanin derivative is melanin attached to a dihydrotrigonelline carrier such as described in Bodor, N., Ann. N. Y. Acad. Sci. 507, 289 (1987), which enables the melanin to cross the blood-brain barrier. The term melanin derivatives is also intended to include chemical derivatives of melanin, such as an esterified melanin.

As used herein, the term “melanin variant” refers to various subsets of melanin substances that occur as families of related materials. Included in these subsets, but not limited thereto, are:
(1) Naturally occurring melanins produced by whole cells that vary in their chemical and physical characteristics;
(2) Enzymatically produced melanins prepared from a variety of precursor substrates under diverse reaction conditions;
(3) Melanin analogs in which a structural feature that occurs in (1) or (2) above is replaced by an unusual substituent divergent from the traditional; and
(4) Melanin derivatives in which a substituent in a melanin produced in (1), (2) or (3) above is further altered by chemical or enzymatic means.

As used herein, the term “Melanin-like substances” refers to heteropolymers of 5-6-dihydroxyindole and 5-6-dihydroxyindole-2-carboxylic acid which have one or more properties usually associated with natural melanins, such as UV absorption or semiconductor behavior.

### Melanin Sources

Melanin and Melanin-like compounds can be obtained:
by extraction and purification from natural sources, e.g. cephalopods such as cuttlefish (e.g. Sepia) or squid (e.g. Loligo), bird feathers (e.g. from species with black strains such as Silkie chickens);
by chemical synthesis, whether water or non-water based e.g. (Deziderio, 2004) (daSilva et al., 2004; Lawrie et al., 2008; Pezzella et al., 2006);
by electrochemical synthesis, e.g. (Meredith et al., 2005);-by bioreactors created by utilization of natural or genetically altered bacteria, fungi, lichens, or viruses e.g. (della-Cioppa, 1998).

Cephalopod inks are natural composites of melanin with other materials, including peptidoglycans, amino acids, proteins, metals, and chemicals and enzymes (such as tyrosinase) which are involved in the synthesis of melanin, and other materials. Cephalopod inks include cuttlefish (such as Sepia), squid, and octopus inks. There is some variation among different species of the percentages of these components. Reports of cephalopod ink components include: Derby, C.D. 2014 Cephalopod Ink: Production, Chemistry, Functions and Applications Marine Drugs 12, 2700-2730; doi: 10.3390/md12052700, and Magarelli M, Passamonti P, Renieri C.

2010. Purification, characterization and analysis of sepia melanin from commercial sepia ink (Sepia Officinalis). Rev CES Med Vet Zootec; Vol 5 (2): 18-28.

### Melanin Manufacturing and Fabrication

Melanin and melanin-like compounds can be manufactured as particles, nanoparticles, dust, beads, or fibers that are woven or non-woven e.g. by methods as described by (Greiner and Wendorff, 2007), sheets e.g. (Meredith et al., 2005), films (daSilva et al., 2004), plates, bricks, chars, spheres, nodules, balls, graphite-like sheets and shards, liquids, gels, or solids (e.g. thermoplastic or thermoset), and by common chemical engineering molding and fabrication methods or custom methods. Sheets can range from one molecular layer to several millimeters. Fibers can range from nanometers to several millimeters.

The melanin material may be natural or synthetic, with natural pigments being extracted from plant and animal sources, such as squid, octopus, mushrooms, cuttlefish, and the like. In some cases, it may be desirable to genetically modify or enhance the plant or animal melanin source to increase the melanin production. Melanins are also available commercially from suppliers.

The following procedure describes an exemplary technique for the extraction of melanin from cuttlefish (Sepia Officinalis). 100 gm of crude melanin are dissected from the ink sac of 10 cuttlefish and washed with distilled water (3×100 ml). The melanin is collected after each wash by centrifugation (200×g for 30 minutes). The melanin granules are then stirred in 800 ml of 8 M Urea for 24 hours to disassemble the melanosomes. The melanin suspension is spun down at 22,000×g for 100 minutes and then washed with distilled water (5×400 ml). The pellet is washed with 50% aqueous DMF (5×400 ml) until a constant UV baseline is achieved from the washes.

Finally, the pellet is washed with acetone (3×400 ml) and allowed to air dry.

Synthetic melanins may be produced by enzymatic conversion of suitable starting materials, as described in more detail hereinbelow. The melanins may be formed in situ within the porous particles or may be performed with subsequent absorption into the porous particles.

Suitable melanin precursors include but are not limited to tyrosine, 3,4-dihydroxy phenylalanine (dopa), D-dopa, catechol, 5-hydroxyindole, tyramine, dopamine, m-aminophenol, oaminophenol, p-aminophenol, 4-aminocatechol, 2-hydroxyl-1,4-naphthaquinone (henna), 4-methyl catechol, 3,4-dihydroxybenzylamine, 3,4-dihydroxybenzoic acid, 1,2-dihydroxynaphthalene, gallic acid, resorcinol, 2-chloroaniline, p-chloroanisole, 2-amino-p-cresol, 4,5-dihydroxynaphthalene 2,7-disulfonic acid, o-cresol, m-cresol, p-cresol, and other related substances which are capable of being oxidized to tan, brown, or black melanin-like compounds capable of absorbing ultraviolet radiation when incorporated in the polymeric particle matrix of the present disclosure. Combinations of precursors can also be used.

The melanin precursor is dissolved in an aqueous solution, typically at an elevated temperature to achieve complete solution. A suitable amount of the enzyme tyrosinase (EC 1.14.18.1) is added to the solution, either before or after the melanin precursor. The concentration of tyrosinase is not critical, typically being present in the range from about 50 to about 5000 U/ml. The solution is buffered with an acetate, phosphate, or other suitable buffer, to a pH in the range from about 3 to 10, usually in the range from about 5 to 8, more usually being about 7. Melanin like pigments can be obtained using suitable precursors even in the absence of an enzyme just by bubbling oxygen through a solution of a precursor for an adequate period of time. Melanin material may be obtained by treatment of, e.g, cuttlefish ink or squid ink in a microwave, optionally with mixing. The inventor has found that microwaving can be used for the preparation of melanin formulations. The compositions and methods as disclosed herein may be produced and practiced using a variety of heating techniques, such as, for example, infrared heating, microwave heating, convection heating, laser heating, sonic heating, or optical heating. For example, it was found that drying melanin in a microwave oven made possible the preparation of large amount of melanin from cuttlefish ink in a very short period of time. In an exemplary embodiment, cuttlefish ink at was placed at 40°° C. in a conventional oven and required 18 days to reduce the material to 40% of its original weight. In a 900 watt microwave oven, the same degree of drying was achieved in 12 minutes.

The disclosure provides a method for formulation of melanin by applying a hydraulic press to melanin partially dried in a microwave oven. In exemplary embodiments, hydraulic presses for this use may range in capacity from, for example, about 1 ton/sq. in. to about 500 tons/in2 approximately. The disclosure provides a method wherein the hydraulic press applies compression of approximately 500 tons/in2. In an exemplary embodiment, commercial cuttlefish ink was dried in a 900 watt microwave oven so that the product was 30% or 35% of the initial weight. A blender was used to mix and grind the melanin. A variety of formulations were made. In one formulation, the 30% preparation was mixed with 7% iron filings, and then the blender was used to mix again. In another formulation, 35% slabs were alternated with 30% slabs to create a layered composite. Each formulation was subjected to compression in a 20 ton/in2 hydraulic press for about 20 minutes. Because the platen was approximately 3.5 in2, it is estimated that a force of approximately 3265 pounds/sq. in. was exerted on each sample formulation.

The disclosure provides for the use of formulations of melanin produced by, for example, microwaving and hydraulic press compression. In an exemplary embodiment, two slabs of melanin were produced by placing cuttlefish ink at 40° C. in a conventional oven and dried for 18 days to reduce the material to 40% of its original weight. In an alternative embodiment, cuttlefish ink was placed in a 900 watt microwave oven, and dried for 12 minutes to form two slabs. Each slab was approximately 3.5 in square. One slab was 1 inch thick and 1 slab was 0.5 in. thick.

The disclosure provides for the use of elemental metals mixed with melanin to create new formulations of melanin with novel properties. The metals may be, for example, iron, copper, zinc, magnesium, manganese, bismuth, calcium, enamel, cesium, radium, strontium, thorium, uranium, or combinations thereof. In an exemplary embodiment, elemental iron was mixed with melanin in the form of dried cuttlefish ink resulted in unexpected hardness of the material while it remains somewhat flexible. Under scanning electron microscopy it was demonstrated that the new formulation of melanin had organized into stacks of lamellae, which appeared to be composed of melanosomes. This is an entirely novel finding since, although metal ions are known to bind to the melanin, it does not appear that anyone has experimented with or reported that elemental iron can bind. This new disclosure is based on the finding that iron and other elemental metals including, for example, copper, zinc, magnesium, manganese, bismuth, calcium, enamel, cesium, radium, strontium, thorium, or uranium, can bind to melanin and organize it in novel ways which confer upon it new properties. For instance, the new properties conferred will include enhanced hardness, stiffness, impact resistance, electrical conductivity, capacitance, semiconductor properties, and enhanced ability to absorb radiation including x-ray and gamma ray.

In an exemplary embodiment, cuttlefish ink was dried using a microwave oven to 40% of its original weight. Iron filings were added so that they comprised 0.5% of the final formulation. The material felt harder than a similar sample without the 0.5% iron filings. Scanning electron microscopy revealed multiple areas where sharply defined lamellae with 90° corner angles were seen in stacks.

The disclosure provides a practical method for formulating melanin to be placed into pharmaceutical or dietary supplement capsules, and other containers. A novel method was developed to enable formulation of melanin (e.g., from cephalopod ink) into capsules or other containers for pharmaceutical, dietary supplement, and other uses. In an exemplary embodiment, cuttlefish ink was dried using a microwave oven to 40% of its original weight. Cab-O-Sil, a pharmaceutical preparation of the excipient micronized silicon dioxide, was mixed to comprise 40% of the final mixture with the 40% dried cuttlefish ink. This mixture was placed in a hard size zero pharmaceutical capsule. After seven days that the capsule became weak and flaccid and would be unsuitable for use. When the mixture of silicon dioxide and cuttlefish ink was dried for several days in a conventional oven at 40° C., then placed in the capsule and observed, the capsule remained intact and is suitable for human and animal use.

In some embodiments, melanins are incorporated into other materials and used for many useful applications, such as:

1. Melanin and melanin-like compounds can be incorporated into: polymers, metals, salts, ceramics of many types, clothing, construction materials, existing armor materials including Kevlar and ceramics, other natural materials or their synthetic mimics, materials for implantation into human or mammalian living beings.

2. A small percentage of melanin confers new or improved properties on resultant material: Another aspect of the present disclosure is that small amounts of melanin and of melanin like substances will impart to a mixture of melanin with other substances, such as a matrix or polymer, properties which are unexpected. Generally, 1 to 5% of melanin will impart desired properties to a mixture or composite, whereas small incremental improvement in properties will be gained by increasing up to 35%. In exemplary embodies as disclosed herein, melanin may be present at about 1%, about 2%, about 3%, about 4%, about 5%, about 6%, about 7%, about 8%, about 9%, about 10%, at a range of about 1% to about 10%, at a range of about 2% to about 8%, at a range of about 3% to about 7%, at a range of about 1% to about 4%, at a range of about 2% to about 5%.

Examples of such unexpected properties are resistance to ultraviolet light, radiation, heat, flame, chemical agents and toxins, biological agents and toxins, and to abrasion.

3. Hydration effects and control: It is another aspect of the present disclosure that the control and maintenance of hydration of melanin and melanin like substances (or non-water solvent or matrix concentration for melanins made from organic solvents) is critical for the applications described above, including armor and shielding. Published research describes the effect of hydration on electrical conductivity, and on the ability to absorb radiation from the electromagnetic spectrum. The present disclosure includes the aspect that when melanin or melanin-like substances are extracted or synthesized, manufactured or fabricated, incorporated in any way with other substances, whether by mixtures, impregnation, layering, compositing, that control and maintenance of desired levels of hydration (and non-water solvent concentration for melanins made from organic solvents) may be critical to achieving and preserving the desired combination of properties. Much of the published research on melanin in the biological, chemical, physics, and electronics literature reports work done using commercially available melanin from Sigma-Aldrich Corp. (St. Louis, Missouri) which is prepared using lyophilization, thus dehydrating it. The present disclosure includes recognition that for the purposes set forth in this disclosure, such as armor and shielding, hydration and control of hydration may be critical for the properties desired in the final material, and the use of highly desiccated or lyophilized melanin may in many instances be undesirable. However, in certain aspects of the disclosure, desiccated or lyophilized melanin may be appropriate.

4. Oxygenation effects and control: It is another aspect of the present disclosure that the control and maintenance of oxygenation, or of lack of access to oxygen, by incorporating melanin into materials that control this factor, or by restricting use to environments that control or restrict this factor, may be critical for certain characteristics to be achieved for shielding, armor, flame retardancy, heat resistance, and cold resistance.

5. Incorporation methods for melanin into other materials includes, for example: mixtures, covalent or non-covalent binding, printing, stamping, electrochemical deposition, metallic salt binding, adhering, and layering in composites. In certain embodiments, the compositions and methods of the disclosure may be produced or practiced using molding techniques such as transfer molding, resin film infusion, resin transfer molding, and structural reaction injection molding (SRIM). In certain embodiments, the compositions and methods of the disclosure may be produced or practiced using molding techniques such as a vacuum assisted resin transfer molding process (VARTM).

In exemplary embodiments, formulations as disclosed herein may comprise active agent at a concentration of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, In exemplary embodiments, formulations as disclosed herein may comprise active agent at a concentration of about 1 to about 20%, of about 5% to about 25%, about 10% to about 20%, or about 15% to about 18%, about 30% to about 70%, about 35% to about 65%, about 63.13%, and about 40% to about 64% w/w.

In an exemplary formulation as disclosed herein, the active agent will represent approximately 1 wt % to 75 wt %, preferably 2 wt % to 30 wt %, more preferably 5 wt. % to 20 wt. % of the total weight.

In other embodiments, the pharmaceutical compositions further comprise one or more additional materials such as a pharmaceutically compatible carrier, binder, viscosity modifier, filling agent, suspending agent, flavoring agent, sweetening agent, disintegrating agent, surfactant, preservative, lubricant, colorant, diluent, solubilizer, moistening agent, stabilizer, wetting agent, anti-adherent, anti-foaming agent, antioxidant, chelating agent, antifungal agent, antibacterial agent, or one or more combination thereof.

### Biological Polymers

The term “biological polymer” according to the disclosure, it is understood collagen and its derivatives, hyaluronic acid, its salts and its derivatives, alginates, synthetic polymers, elastin and biological polymers, and mixtures thereof. Preferably, the biological polymer may comprises compounds chosen from collagen, collagen of porcine origin, collagen of bovine origin, crosslinked collagens, hyaluronic acid, its salts and its derivatives, lactic acid polymers, methacrylate derivatives, calcium phosphate derivatives, polyacrylamides, polyurethanes, polyalkylimide gels, polyvinyl microspheres, silicones, silica (SiO2) polymers, and mixtures thereof.

Collagen is a fibrous protein, of approximately 300 kDa, which makes up the connective tissue in the animal kingdom. It may be of human or nonhuman origin, in particular of porcine or bovine origin. Collagen derivatives include, inter alia, crosslinked collagens.

The composites of the disclosure may be formed from a wide variety of polymers, including natural polymers such as carboxylmethylcellulose, cellulose acetate phthalate, ethylcellulose, methylcellulose, arabinogalactan, nitrocellulose, propylhydroxycellulose, and succinylated gelatin; and synthetic polymers such as polyvinyl alcohol, polyethylene, polypropylene, polystyrene, polyacrylamide, polyether, polyester, polyamide, polyurea, epoxy, ethylene vinyl acetate copolymer, polyvinylidene chloride, polyvinyl chloride, polyacrylate, polyacrylonitrile, chlorinated polyethylene, acetal copolymer, polyurethane, polyvinyl pyrrolidone, poly (p-xylene), polymethylmethacrylate, polyvinyl acetate, polyhydroxyethyl methacrylate, and combinations thereof.

### Utility and Characteristics

The following characteristics and functions for armor, shielding and other applications can be achieved, in almost infinite variety of degrees and combinations:

### Protection from Weapons

It is another aspect of the present disclosure that melanin and composite materials incorporating melanin can be used for shielding from biological, chemical, radiological and nuclear weapons.

It is another aspect of the present disclosure that melanin and composites materials incorporating melanin can be used for shielding from impact due to bullets or other projectiles or explosives, including shaped charges.

The current disclosure is directed to a ballistic protection material composition comprising one or more type of, e.g., ceramic powders or particles mixed with one or more type of melanin materials. In one embodiment, in addition to the melanin material, other polymeric materials may be further selected from the group consisting of rigid-rod polymers, semi-rigid-rod polymers, polyimides, polyetherimides, polyimideamides, polysulfones, epoxy resins, bismaleimide resins, bis-benzocyclobutene resins, phthalonitrile resins, polyaryletherketones, polyetherketones, liquid crystal polymers, oligomeric cyclic polyester precursors, polybenzbisoxazoles, polybenzbisthiazoles, polybenzbisimidazoles, acetylene endcapped thermosetting resins, PrimoSpire® polymers, polysulfones, polyaramides, poly-paraphenylene terephthalamide, polyamides, polycarbonates, polyethylenes, polyesters, polyphenols and polyurethanes.

In another embodiment, the composition further comprises one or more types of process aids, modifiers, colorants, fibers, adhesion promoters and fillers.

In still another embodiment, ceramic powders or particles are selected from the group consisting of alumina, boron carbide, boron nitride, mullite, silica, silicon carbide, silicon nitride, magnesium boride, multi-walled carbon nanotubes, single walled carbon nanotubes, group IVB, VB and VIB metal sulfide nanotubes, titanium boride, titanium carbide, and diamond.

In yet another embodiment, ceramic powders or particles provide 10% to 98% of the total mass, in a preferred embodiment the ceramic powders or particles provide 20% to 95% of the total mass, and in a most preferred embodiment the ceramic powders or particles provide at least 50% of the total mass.

In still yet another embodiment, ceramic powders or particles have particle size in the range of 10 nanometers to 100 microns; and in a preferred embodiment the ceramic powders or particles have particle size in the range of 100 nanometers to 10 microns.

In still yet another embodiment, the melanin material or materials provide 2% to 95% of the total mass, and in a preferred embodiment the melanin material or materials provide less than 50% of the total mass.

In still yet another embodiment, the ballistic protection materials are used together with other ballistic materials, including, but not limited to woven ballistic fabrics (such as but not limited to polyaramid or polyethylene fabrics), metals, ceramics, and the like. In still yet another embodiment, the ballistic protection materials are incorporated into an article selected from the group consisting of: a ballistic protection article, a helmet, a sheet or panel, such as a vehicle or blast protection panel, body armor, and cargo containers.

### Protection from Lasers

It is another aspect of the present disclosure that melanin and composite materials including melanin can be used for shielding from lasers.

### Thermal Properties

Melanin's ability to resist degradation by extreme heat, e.g. >500° C., was reported by Deziderio (Deziderio, 2004). Melanin's ability to resist degradation by extreme cold (slightly above absolute zero) was reported by (Yang and Anderson, 1986). The present disclosure includes the discovery that melanin can be used alone, or in composites with other materials such as metals and polymers, to resist destruction by high heat or temperature, for shielding, armor, and aerospace applications such as airplane and space vehicle construction parts.

### Chemical Properties

The ability of melanin to resist degradation by chemicals of all types, including strong acids (such as hydrochloric acid) and bases (such as sodium hydroxide), was reviewed by (Prota, 1992). The present disclosure includes the discovery that melanin can be used alone, or in composites with other materials such as metals and polymers, to resist destruction by chemicals including strong acids and strong bases, for shielding, armor, and aerospace applications such as airplane and space vehicle construction parts.

### Protection from Radiation

It has been reported that melanin absorbs beta particles, gamma rays, X-rays, infrared, visible, ultraviolet, the remainder of the electromagnetic spectrum, and combinations thereof. The present disclosure includes the discovery that melanin can be used alone, or in composites with other materials such as lead and polymers, to absorb and prevent destruction by radiation, e.g., for shielding, armor, and aerospace applications such as airplane and space vehicle construction parts.

The present disclosure includes the discovery that radioprotectant/radiomitigation hybrid compositions, such as bismuth-melanin composites can be used alone, or in composites with other materials for:
a. shielding of radiation from sources like uranium and radium.
b. to degrade, encapsulate and shield from living and non-living radioactive particles in sizes from nanometers to millimeters.
c. to shield personnel and equipment from radiation from depleted uranium used in weaponry or armor.

The present disclosure includes the discovery that bismuth-melanin can be used alone, or in composites with other materials not only by covering a human or other organism by bismuth-melanin, alone or in mixture with other materials: It can be accomplished by ingestion, injection, or other internal administration of these compounds or composites.

Furthermore, the bismuth-melanin, can be used to mitigate the destructive biological effects of radiation, even if the radiation has been absorbed. For instance, radiation creates free radicals in biological tissues which creates great damage in the hematopoietic and gastrointestinal systems. Bismuth-melanin is known to absorb such free radicals and mitigate such damage.

### Binding to Metals and Radioactive Substances

It has been reported that melanin binds to metals and radioactive substances (Bruenger et al., 1967) (Fogarty and Tobin, 1996) (Kasatna et al, 2003) (Taylor et al., 1964). The present disclosure includes the discovery that melanin can be used alone, or in composites with other materials to form shielding and armor and for aerospace applications, specifically because it naturally binds to a wide range of metals and to radioactive substances.

### Binder

Binders are useful in fabricating materials from non or loosely assembled matter. For example, binders enable two or more surfaces to become united. In certain embodiments, non-melanin material may be included in the compositions and methods of the disclosure and may be a binder. In exemplary embodiments, any adhesive material, such as phenolic resins, ureaformaldehyde resins, melamine formaldehyde resins, hyde glue, aminoplast resins, epoxy resins, acrylate resins, latexes, polyester resins, urethane resins, and mixtures thereof may be used as a binder. Suitable binders include glue, varnish, epoxy resins, phenolic resins, polyurethane resins. In exemplary embodiments, the binder may be, for example, glue, which may be selected from the group consisting of Clear Weld, LOCTITE® Heavy Duty Epoxy, LOCTITE® Epoxy Metal/Concrete, LOCTITE INSTANT-MIX®, LOCTITE®, LOCTITE® BULLDOG, LOCTITE® PL Marine Adhesive Sealant, E6000®, (E6000 STITCHLESS®, E6000 EXTREME TACK®, E6000 FABRI-FUSE®, PRO-POXY® 20, TITEBOND III®, TITEBOND III ULTIMATE WOOD GLUE®, FIBER FIX SUPER TAPE, ELMER'S SCHOOL GLUE NATURALS®, ELMER'S GLUE-ALL®, Elmer's Multi Purpose All Glue, KRAZY GLUE®, LIQUID NAILS®, PRODUTY® HEAVY DUTY CONSTRUCTION ADHESIVE, Firmo Liquid, Welbond Universal Adhesive, and combinations thereof.

Thermally curable resins suitable for use in accordance with the compositions and methods of the disclosure are preferably selected from the group consisting of phenolic resins, urea formaldehyde resins, melamine-formaldehyde resins, epoxy resins, acrylate resins, urethane resins, melamine resins, alkyd resins, and polyimide resins, isocyanate, isocyanurate, and combinations thereof. Multifunctional acrylates are preferably selected from trimethylolpropane triacrylate, glycerol triacylate, pentaerythritol triacrylate and methacrylate, pentaerythritol tetraacrylate and methacrylate, dipentaerythritol pentaacrylate, sorbitol triacrylate, and sorbital hexaacrylate.

Thermoplastic binders comprise a variety of polymerized materials such as polyvinyl acetate, polyvinyl butyral, polyvinyl alcohol, and other polyvinyl resins; polystyrene resins; acrylic and methacrylic acid ester resins; cyanoacrylates; and various other synthetic resins such as polyisobutylene polyamides, courmarone-idene products, and silicones.

Suitable functionalized acrylics, alkyds, polyurethanes, polyesters, and epoxies can be obtained from a number of commercial sources. Useful acrylics are sold under the ACRYLOID™ trade name (Rohm & Haas, Co., Pennsylvania); useful epoxy resins are sold under the EPON™ trade name (Resolution Specialty Materials, LLC, Illinois); useful polyester resins are sold under the CYPLEX® trade name (Cytec Industries, New Jersey); and useful vinyl resins are sold under the UCAR™ trade name (The Dow Chemical Company, Michigan).

Illustrative of useful high modulus or rigid binder materials are polycarbonates; polyphenylenesulfides; polyphenylene oxides; polyester polyesterimides; polyimides; and thermoset resins such as epoxy resins, phenolic resins, modified phenolic resins, allylic resins, alkyd resins, unsaturated polyesters, aromatic vinylesters as for example the condensation produced of bisphenol A and methacrylic acid diluted in a vinyl aromatic monomer (e.g. styrene or vinyl toluene), urethane resins and amino (melamine and urea) resins. The major criterion is that such material holds the composition together and maintains the geometrical integrity of the composite under the desired use conditions. The binder can be included in the composition in any suitable amount. For example, the binder can be included in an amount from about 5 wt. % to about 100 wt. % by weight (on a solids basis) of the wet composition, such as from about 20 wt. % to about 80 wt. %, from about 30 wt. % to about 70 wt. %, from about 40 wt. % to about 60 wt. %, etc.

### Pharmaceutical Formulations

The disclosure provides a practical method for formulating compositions comprising melanin to be placed into pharmaceutical or dietary compositions. The disclosure provides a practical method for formulating melanin to be placed into pharmaceutical or dietary supplement capsules, and other containers. A novel method was developed to enable formulation of melanin (e.g., from cephalopod ink) into capsules or other containers for pharmaceutical, dietary supplement, and other uses. In an exemplary embodiment, cuttlefish ink was dried using a microwave oven to 40% of its original weight. Cab-O-Sil, a pharmaceutical preparation of the excipient micronized silicon dioxide, was mixed to comprise 40% of the final mixture with the 40% dried cuttlefish ink. This mixture was placed in a hard size zero pharmaceutical capsule. After seven days that the capsule became weak and flaccid and would be unsuitable for use. When the mixture of silicon dioxide and cuttlefish ink was dried for several days in a conventional oven at 40° C., then placed in the capsule and observed, the capsule remained intact and is suitable for human and animal use.

The compositions of the disclosure may be administered enterally or parenterally. Mixed with pharmaceutically suitable auxiliaries, e.g., as described in the standard reference, Gennaro et al., Remington's Pharmaceutical Sciences. The compositions may be compressed into solid dosage units, such as pills, tablets, or be processed into capsules or suppositories. By means of pharmaceutically suitable liquids the compositions can also be applied in the form of a solution, suspension, emulsion, e.g. for use as an injection preparation or eye drops, or as a spray, e.g. for use as a nasal spray.

For making dosage units, e.g., tablets, the use of conventional additives such as fillers, colorants, polymeric binders and the like is contemplated. In general, any pharmaceutically acceptable additive which does not interfere with the function of the active compounds can be used. Suitable carriers with which the compositions can be administered include lactose, starch, cellulose derivatives and the like, or mixtures thereof, used in suitable amounts.

Active Agent

In exemplary embodiments, formulations as disclosed herein may comprise active agent at a concentration of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, In exemplary embodiments, formulations as disclosed herein may comprise active agent at a concentration of about 1 to about 20%, of about 5% to about 25%, about 10% to about 20%, or about 15% to about 18%, about 30% to about 70%, about 35% to about 65%, about 63.13%, and about 40% to about 64% w/w.

In certain embodiments the active agent is present as a highly purified extract of active agent which comprises at least 90%, 91%, 92%, 93%, 94%, 95%, 96%, 97%, 98%, 99%, 99.5%, or 99.75% (w/w) of the formulation.

In certain embodiments the active agent is present in the formulation provided at a concentration of at least 90%, 91%, 92%, 93%, 94%, 95%, 96%, 97%, 98%, 99%, 99.5%, or 99.75%, or 100% (w/w).

In certain embodiments the active agent is 100% synthetic. In certain embodiments the active agent has a purity equal to or greater than about 90%, 91%, 92%, 93%, 94%, 95%, 96%, 97%, 98%, 99%, 99.5%, 99.75%, or 100% (w/w). In certain embodiments the active agent is produced synthetically and has a purity equal to or greater than about 90%, 91%, 92%, 93%, 94%, 95%, 96%, 97%, 98%, 99%, 99.5%, 99.75%, or 100% (w/w). In certain embodiments the active agent is a combination of active agents, and each active agent may be produced synthetically and independently have a purity equal to or greater than about 90%, 91%, 92%, 93%, 94%, 95%, 96%, 97%, 98%, 99%, 99.5%, 99.75%, or 100% (w/w).

In certain embodiments, the dose of active agent is equal to or greater than, for example, about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, or 45 mg/kg/day. In certain embodiments, the dose of active agent is equal to or greater than, for example, about 0.001 ng/day, 0.01 ng/day, 0.025 ng/day. 0.05 ng/day, 0.1 ng/day, 0.25 ng/day, 0.5 ng/day, 1 ng/day, 10 ng/day, 25 ng/day, 50 ng/day, 100 ng/day, 250 ng/day, 500 ng/day, 1000 ng/day, 0.001 microgram/day, 0.01 microgram/day, 0.025 microgram/day, 0.050 microgram/day, 0.1 microgram/day, 0.25 microgram/day, 0.5 microgram/day, 1 microgram/day, 2.5 microgram/day, 5 microgram/day, 10 microgram/day, 25 microgram/day, 50 microgram/day, 100 microgram/day, 250 microgram/day, or 500 microgram/day. In certain embodiments, the dose of active agent is equal to or greater than, for example, about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 ng/day. In certain embodiments, the dose of active agent is equal to or greater than, for example, about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 mg/day. In exemplary embodiments, formulations of the disclosure may comprise active agent at a concentration of about 0.001 ng, 0.01 ng, 0.025 ng. 0.05 ng, 0.1 ng, 0.25 ng, 0.5 ng, 1 ng, 10 ng, 25 ng, 50 ng, 100 ng, 250 ng, 500 ng, 1000 ng, 0.001 microgram, 0.01 microgram, 0.025 microgram. 0.05 microgram, 0.1 microgram, 0.25 microgram, 0.5 microgram, 1 microgram, 2.5 microgram, 5 microgram, 10 microgram, 25 microgram, 50 microgram, 100 microgram, 250 microgram, or 500 microgram. In exemplary embodiments, formulations of the disclosure may comprise active agent at a concentration of about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 ng. In exemplary embodiments, formulations of the disclosure may comprise active agent at a concentration of about 0.001, 0.0025 0.005, 0.0075, 0.01, 0.025, 0.05, 0.075, 0.1, 0.25, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 mg.

In an exemplary formulation as disclosed herein, the active agent will represent approximately 1 wt % to 75 wt %, preferably 2 wt % to 30 wt %, more preferably 5 wt. % to 20 wt. % of the total weight.

In other embodiments, the pharmaceutical compositions further comprise one or more additional materials such as a pharmaceutically compatible carrier, binder, viscosity modifier, filling agent, suspending agent, flavoring agent, sweetening agent, disintegrating agent, surfactant, preservative, lubricant, colorant, diluent, solubilizer, moistening agent, stabilizer, wetting agent, anti-adherent, parietal cell activator, anti-foaming agent, antioxidant, chelating agent, antifungal agent, antibacterial agent, or one or more combination thereof.

### Therapy

Any therapy (e.g., therapeutic or prophylactic agent) which is useful, has been used, is currently being used, or may be used for the prevention, treatment and/or management of radiation exposure can be used to prevent, treat, and/or manage a patient with the compositions and methods as disclosed herein, for example with compositions and methods for the oral administration of a composition comprising radioprotectant/radiomitigation hybrid compositions as disclosed herein.

Currently available therapies and their dosages, routes of administration and recommended usage are known in the art and have been described in such literature as the Physician's Desk Reference (60th ed., 2006).

In a specific embodiment, cycling therapy involves the administration of a first therapeutic for a period of time, followed by the administration of a second therapeutic for a period of time, optionally, followed by the administration of a cancer therapeutic for a period of time and so forth, and repeating this sequential administration, i.e., the cycle in order to reduce the development of resistance to one of the therapeutics, to avoid or reduce the side effects of one of the therapeutics, and/or to improve the efficacy of the therapeutics.

When two prophylactically and/or therapeutically effective regimens are administered to a subject concurrently, the term “concurrently” is not limited to the administration of the therapeutics at exactly the same time, but rather, it is meant that they are administered to a subject in a sequence and within a time interval such that they can act together (e.g., synergistically to provide an increased benefit than if they were administered otherwise). For example, the therapeutics may be administered at the same time or sequentially in any order at different points in time; however, if not administered at the same time, they should be administered sufficiently close in time so as to provide the desired therapeutic effect, preferably in a synergistic fashion. The combination cancer therapeutics can be administered separately, in any appropriate form and by any suitable route. When the components of the combination therapeutics are not administered in the same pharmaceutical composition, it is understood that they can be administered in any order to a subject in need thereof. For example, a first prophylactically and/or therapeutically effective regimen can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second cancer therapeutic, to a subject in need thereof. In various embodiments, the cancer therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the cancer therapeutics are administered within the same office visit. In another embodiment, the combination cancer therapeutics are administered at 1 minute to 24 hours apart.

### Carrier

Examples of lipids that may be employed in the compositions and methods as disclosed herein include, but are not limited to, fats, oils, waxes, fatty acids, fatty acid esters, glycerides, fatty alcohols, hydrogenated vegetable oil, soybean oil, phospholipids, terpenes and the like or combinations thereof. Suitable waxes that may be employed include, but are not limited to, natural waxes, such as animal waxes, vegetable waxes, and petroleum waxes (i.e., paraffin waxes, microcrystalline waxes, petrolatum waxes, mineral waxes), and synthetic waxes. Non-limiting examples include, but are not limited to, spermaceti wax, carnauba wax, Japan wax, bayberry wax, flax wax, beeswax, Chinese wax, shellac wax, lanolin wax, sugarcane wax, candelilla wax, paraffin wax, microcrystalline wax, petrolatum wax, carbowax, and the like, or mixtures thereof. Mixtures of these waxes with the fatty acids may also be used. Non-limiting examples of oils that may be employed include, castor oil, soybean oil, and the like or combinations thereof. Fatty acids that may be employed in the present invention include, but are not limited to, decenoic acid, docosanoic acid, stearic acid, palmitic acid, lauric acid, myristic acid, and the like, and mixtures thereof. Suitable fatty alcohols that may be employed in the compositions as disclosed herein include, but are not limited to, cetyl alcohol, stearyl alcohol or mixtures thereof. Suitable hydrogenated vegetable oils that may be employed in the compositions as disclosed herein, include but are not limited to, hydrogenated palm kernel oil, hydrogenated peanut oil, hydrogenated palm oil, hydrogenated rapeseed oil, hydrogenated rice bran oil, hydrogenated soybean oil, hydrogenated sunflower oil, hydrogenated castor oil, hydrogenated cottonseed oil, and the like, and mixtures thereof. In one embodiment, lipids may be employed in case the carrier particles being prepared are solid lipid nanoparticles, lipid-based nanoparticles or microparticles, nanoemulsions, microemulsions, liposomes, and the like or combinations thereof.

### Dosage Forms

The compositions as disclosed herein can provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a granule, a film, a sprinkle, a pellet, a bead, a pill, a powder, a triturate, a platelet, a strip or a sachet. Compositions can also be administered after being mixed with, for example yoghurt or fruit juice and swallowed or followed with a drink or beverage. These forms are well known in the art and are packaged appropriately. The compositions can be formulated for oral or rectal delivery.

Tablets prepared for oral administration according to the invention, and manufactured using direct compression, will generally contain other inactive additives such as binders, lubricants, disintegrants, fillers, stabilizers, surfactants, coloring agents, and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate ( ), calcium stearate, stearic acid, and hydrogenated vegetable oil (preferably comprised of hydrogenated and refined triglycerides of stearic and palmitic acids at about 1 wt. % to 5 wt. %, most preferably less than about 2 wt. %). Lubricants may be present in a concentration of, for example, from about 0.25 wt. % to about 3 wt. %, 0.5 wt. % to about 2.0 wt. %, from about 0.75% to about 1.5%.

Disintegrants are used to facilitate disintegration of the tablet, thereby increasing the erosion rate relative to the dissolution rate, and are generally starches, clays, celluloses, algins, gums, or crosslinked polymers (e.g., crosslinked polyvinyl pyrrolidone). Fillers include, for example, materials such as silicon dioxide, titanium dioxide, alumina, talc, kaolin, powdered cellulose, and microcrystalline cellulose, as well as soluble materials such as mannitol, urea, sucrose, lactose, lactose monohydrate, dextrose, sodium chloride, and sorbitol. Solubility-enhancers, including solubilizers per se, emulsifiers, and complexing agents (e.g., cyclodextrins), may also be advantageously included in the present formulations. Stabilizers, as well known in the art, are used to inhibit or retard drug decomposition reactions that include, by way of example, oxidative reactions. Disintegrants may be present in a concentration of, for example, from about 0.25 wt. % to about 3 wt. %, 0.5 wt. % to about 2.0 wt. %, from about 0.75% to about 1.5%.

Shellac, also called purified lac, a refined product obtained from the, resinous secretion of an insect. This coating dissolves in media of pH>7.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

In carrying out the method as disclosed herein, the combination of the invention may be administered to mammalian species, such as dogs, cats, humans, etc. and as such may be incorporated in a conventional systemic dosage form, such as a tablet, capsule, or elixir. The above dosage forms will also include the necessary carrier material, excipient, viscosity modifier, lubricant, buffer, antibacterial, bulking agent (such as mannitol), anti-oxidants (ascorbic acid of sodium bisulfate) or the like.

The dose administered may be carefully adjusted according to age, weight and condition of the patient, as well as the route of administration, dosage form and regimen and the desired result.

The compositions of the invention may be administered in the dosage forms in single or divided doses of one to four times daily, or may be administered multiple times per day. It may be advisable to start a patient on a low dose combination and work up gradually to a high dose combination.

Tablets of various sizes can be prepared, e.g., of about 2 to 2000 mg in total weight, containing one or both of the active ingredients, with the remainder being a physiologically acceptable carrier of other materials according to accepted practice. Gelatin capsules can be similarly formulated.

Liquid formulations can also be prepared by dissolving or suspending one or the combination of active substances in a conventional liquid vehicle acceptable for administration so as to provide the desired dosage in, for example, one to four teaspoonfuls.

Dosage forms can be administered to the patient on a regimen of, for example, one, two, three, four, five, six, or other multiple doses per day.

In order to more finely regulate the dosage schedule, the active substances may be administered separately in individual dosage units at the same time or carefully coordinated times. The respective substances can be individually formulated in separate unit dosage forms in a manner similar to that described above.

In formulating the compositions, the active substances, in the amounts described above, may be compounded according to accepted practice with a physiologically acceptable vehicle, carrier, excipient, binder, viscosity modifier, preservative, stabilizer, flavor, etc., in the particular type of unit dosage form.

### Packaging/Treatment Kits

The disclosure provides a kit for conveniently and effectively carrying out the methods in accordance with the present disclosure. Such kits may be suited for the delivery of solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging unit dosage forms. If desired, the blister can be in the form of a childproof blister, i.e. a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as, for example, an AM dose is packaged with a “midday” and a PM dose.; or an AM dose is packaged with a PM dose. Alternatively, placebo dosages, or vitamin or dietary supplements, either in a form similar to or distinct from the active dosages, can be included.

The disclosure provides compositions, including preparations, formulations and/or kits, comprising combinations of ingredients, as described above (including the multi-ingredient combinations of drugs of the invention), that are serviceable as therapies for treating, preventing or improving conditions, states and disease as provided in the invention. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package consists two or more separate compartments. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the agents of the invention. In one aspect, a blister pack of the invention comprises a molded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the invention are contained in-between a card and clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a combination of the invention), and then a “blister card” that is a printed card with an adhesive coating on the front surface. During the assembly process, the blister component, which is most commonly made out of PVC, is attached to the blister card using a blister machine. Conventional blister packs can also be sealed.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

In one aspect, any of the invention's products of manufacture, including kits or blister packs, include memory aids to help remind patients when and how to take the agents of the invention.

The treatment kits can be constructed in a variety of forms familiar to one of ordinary skill in the art. The kits comprise at least one unit dosage of an active for administration according to a daily regimen and a means for containing the unit dosages. The treatment kits can, for example, be constructed for administration once daily, twice daily, thrice daily, four times daily, multiple administrations daily, or other dosage regimens. The kits comprise a means for the daily administration of an agent of the invention. In one embodiment the kits include from about one to about four unit dosages.

In one embodiment, the means for containing the unit dosages is a card, including, for example, a card that is capable of being folded. This card will be referred to herein as a main card, or alternatively a principal card or a first card, to distinguish it from additional optional cards, circulars, or other such materials which can be associated with the kit. This main card can be folded with a simple crease, or alternatively, with a double crease, so as to exhibit a spine, similar to the spine of a closed book. The main card can comprise a printable surface, i.e. a surface upon which the product name, appropriate administration instructions, product information, drawings, logos, memory aids, calendar features, etc. can be printed. The main card can comprise a means for containing said unit dosage or different dosages designated for different time of the day, and a memory aid for administering said unit dosage or dosages. The main card, especially if it is prepared from two or more laminated paperboard surfaces, can comprise a slit or pocket, for example in one of the inner paperboard surfaces of the folded card. The slit or pocket can be used to contain a removable secondary card, i.e., a second card or insert card, which is not permanently attached or affixed to the main card.

The memory aid can include a listing of the days of the week, i.e. Sunday, Monday, Tuesday, Wednesday, Thursday, Friday, and Saturday, with appropriate spaces for the patient to select and indicate on the card the preferred day of the week on which to administer the therapy. The memory aid can include a listing of the time of day with appropriate spaces for the patient to select and indicate on the card the preferred time of day (e.g.: AM, PM, midday) at which to administer the therapy. The memory aid can also include removable stickers having an appropriate pressure sensitive adhesive to facilitate easy removal and refastening to a desired surface such as a calendar or dayminder. The removable stickers can be located on the main card, or can be located on the secondary card which is constructed so that it can be readily inserted into and removed from the optional slit in the main card. Additionally, the optional slit can contain additional patient information and other circulars.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

The invention will be illustrated in more detail with reference to the following Examples, but it should be understood that the present invention is not deemed to be limited thereto.

### EXAMPLES

### Example 1

### Melanin as a Decorporation Material for Radioactive Strontium

Strontium is a frequent contaminant resulting from nuclear accidents. Strontium has the following stable non-radioactive isotopes: ⁸⁴Sr, ⁸⁶Sr, ⁸¹Sr, ⁸⁸Sr The radioactive isotopes of strontium are included in the list of ⁷³Sr to ¹⁰⁸Sr (e.g. There are about 32 radioactive isotopes). Strontium-90 was distributed due to the Chernobyl disaster. Binding experiments and kinetic evaluations are carried out with non-radioactive strontium. All the results are applicable to radioactive strontium.

### Example 2

### Melanin as a Decorporation Material for Radioactive Cobalt

Cobalt is of concern because it is possible for enemies to construct a cobalt bomb (Geist, E. M. 2016 Would Russia's undersea “doomsday drone” carry a cobalt bomb? Bulletin Of The Atomic Scientists, 2016 VOL. 72, NO. 4, 238-242 http://dx.doi.org/10.1080/00963402.2016.1195199) and because accidents involving contamination of radioactive cobalt have occurred. Cobalt has the following stable non-radioactive isotope: ⁵⁹Co There are a total of 28 radioisotopes of cobalt. Cobalt-60 is of the most practical concern. Binding experiments and kinetic evaluations are carried out with non-radioactive cobalt All the results are applicable to radioactive cobalt.

While the invention has been described in detail and with reference to specific examples thereof, it will be apparent to one skilled in the art that various changes and modifications can be made therein without departing from the spirit and scope thereof.

What is claimed is:

1. A radioprotectant/radiomitigation hybrid pharmaceutical composition comprising:
at least one therapeutically effective amount of a radiation absorbing component;
at least one therapeutically effective amount of a decorporation material component; and at least one pharmaceutically acceptable excipient.

2. The radioprotectant/radiomitigation hybrid pharmaceutical composition of claim 1 wherein the radiation absorbing component is selected from the group consisting of a biological material, melanin, melanin which has been doped with additives, melanin which has been doped with at least one metal, and combinations thereof.

3. The radioprotectant/radiomitigation hybrid pharmaceutical composition of claim 1 wherein the radiation absorbing component is melanin which has been doped with at least one metal, wherein the metal is selected from the group consisting of iron, copper, zinc, magnesium, manganese, bismuth, calcium, enamel, cesium, radium, strontium, thorium, uranium, and combinations thereof.

4. The radioprotectant/radiomitigation hybrid pharmaceutical composition of claim 1 wherein the decorporation material component is selected from the group consisting of a biological pigment, melanin, and combinations thereof.

5. The radioprotectant/radiomitigation hybrid pharmaceutical composition of claim 1 wherein the decorporation material component absorbs, adsorbs, or binds in any manner to radioactive substances selected from the group consisting of ²²⁵Actinium, ²²⁷Actinium, ¹³³Barium, ²¹³Bismuth, ¹³⁷Cesium, ⁶⁰Cobalt¹¹¹Indium, ²⁰⁹Lead, ²²⁴Radium, ⁸⁵Strontium, ²²⁸Thorium, Uranium, ⁹⁰Yttrium, combinations thereof, beta particles, gamma rays, X-rays, infrared, visible, ultraviolet, the remainder of the electromagnetic spectrum, and metals or other undesirable substances that contaminate organisms, and combinations thereof.

6. The radioprotectant/radiomitigation hybrid pharmaceutical composition of claim 1 wherein the radioprotectant/radiomitigation hybrid pharmaceutical composition is in the form of a capsule filled with nanoparticles, wherein for each nano particle the core is the radiation absorber, and the shell is the decorporation material.

7. The radioprotectant/radiomitigation hybrid pharmaceutical composition of claim 1 wherein the radioprotectant/radiomitigation hybrid pharmaceutical composition is in a form that can be injected or instilled or implanted into a specific organ.

8. The radioprotectant/radiomitigation hybrid pharmaceutical composition of claim 1 wherein the radioprotectant/radiomitigation hybrid pharmaceutical composition is in a form that can be instilled into the lungs, using bronchoscopy.

9. The radioprotectant/radiomitigation hybrid pharmaceutical composition of claim 1 wherein the radioprotectant/radiomitigation hybrid pharmaceutical composition is in a form that can be delivered into the trachea using catheters.

10. The radioprotectant/radiomitigation hybrid pharmaceutical composition of claim 1 wherein the radioprotectant/radiomitigation hybrid pharmaceutical composition is in a form that can be injected or instilled or implanted into a catheter could be placed in a portal vein so that the device or therapy would be immediately distributed to the liver.

11. The radioprotectant/radiomitigation hybrid pharmaceutical composition of claim 1 wherein the radiation absorber layer and the decorporation layer will remain bound together in the body.

12. The radioprotectant/radiomitigation hybrid pharmaceutical composition of claim 1 wherein the radiation absorber layer and the decorporation layer will dissociate in the body.

13. The radioprotectant/radiomitigation hybrid pharmaceutical composition of claim 1 wherein the radiation absorber layer and the decorporation layer are in the form of a central cylinder radiation absorber layer, and an outer coil made of the decorporation material, optionally wherein these two layers separate in the body.

14. The radioprotectant/radiomitigation hybrid pharmaceutical composition of claim 1 wherein the radioprotectant/radiomitigation hybrid pharmaceutical composition is a capsule filled with nanoparticles, that can be swallowed, and the nanoparticles dispersed throughout the body.

15. The radioprotectant/radiomitigation hybrid pharmaceutical composition of claim 1 wherein the radiation absorber layer also has decorporation properties.

16. The radioprotectant/radiomitigation hybrid pharmaceutical composition of claim 1 wherein the radioprotectant/radiomitigation hybrid pharmaceutical composition is in a form selected from the group consisting of as a spherical, oval, or cylindrical, or rectangular shape which has a inner (core) component which is the radiation absorber and an outer (shell) component which is the decorporation material.

17. A method for the prevention, treatment and/or management of radiation exposure in a subject in need thereof comprising:
selecting a subject in need of prevention, treatment and/or management of radiation exposure;
administering to the subject the radioprotectant/radiomitigation hybrid pharmaceutical composition of claim 1,
wherein the administration of the radioprotectant/radiomitigation hybrid pharmaceutical composition prevents, treats, and/or manages radiation exposure in the subject.

18. The method of claim 17, wherein the radiation exposure is a radioactive substances selected from the group consisting of ²²⁵Actinium, ²²⁷Actinium, ¹³³Barium, ²¹³Bismuth, ¹³⁷Cesium, ⁶⁰Cobalt¹¹¹Indium, ²⁰⁹Lead, ²²⁴Radium, ⁸⁵Strontium, ²²⁸Thorium, Uranium, ⁹⁰Yttrium, combinations thereof, beta particles, gamma rays, X-rays, infrared, visible, ultraviolet, the remainder of the electromagnetic spectrum, and metals or other undesirable substances that contaminate organisms, and combinations thereof.

19. The method for the prevention, treatment and/or management of radiation exposure in a subject in need thereof of claim 17 wherein the radioprotectant/radiomitigation hybrid pharmaceutical composition is in the form of a capsule filled with nanoparticles, wherein for each nano particle the core is the radiation absorber, and the shell is the decorporation material.

20. The method for the prevention, treatment and/or management of radiation exposure in a subject in need thereof of claim 17 wherein the radioprotectant/radiomitigation hybrid pharmaceutical composition is in a form that could be injected or instilled or implanted into a specific organ.

21. The method for the prevention, treatment and/or management of radiation exposure in a subject in need thereof of claim 17 wherein the radioprotectant/radiomitigation hybrid pharmaceutical composition is in a form that can be instilled into the lungs, using bronchoscopy.

22. The method for the prevention, treatment and/or management of radiation exposure in a subject in need thereof of claim 17 wherein the radioprotectant/radiomitigation hybrid pharmaceutical composition is in a form that can be delivered into a trachea using catheters.

23. The method for the prevention, treatment and/or management of radiation exposure in a subject in need thereof of claim 17 wherein the radioprotectant/radiomitigation hybrid pharmaceutical composition is in a form could be injected or instilled or implanted into a catheter can be placed in the portal vein so that the radioprotectant/radiomitigation hybrid pharmaceutical composition would be immediately distributed to the liver.

24. The method for the prevention, treatment and/or management of radiation exposure in a subject in need thereof of claim 17 wherein the radiation absorber layer and the decorporation layer will remain bound together in the body.

25. The method for the prevention, treatment and/or management of radiation exposure in a subject in need thereof of claim 17 wherein the radiation absorber layer and the decorporation layer will dissociate in the body.

26. The method for the prevention, treatment and/or management of radiation exposure in a subject in need thereof of claim 17 wherein the radiation absorber layer and the decorporation layer are in the form of a central cylinder radiation absorber layer, and an outer coil made of the decorporation material, optionally wherein these two layers separate in the body.

27. The method for the prevention, treatment and/or management of radiation exposure in a subject in need thereof of claim 17 wherein the radioprotectant/radiomitigation hybrid device is a capsule filled with nanoparticles, that is be swallowed and the nanoparticles dispersed throughout the body.

28. The method for the prevention, treatment and/or management of radiation exposure in a subject in need thereof claim 17 wherein the radiation absorber layer also has decorporation properties.

29-86. (canceled)
